(19) United States
(12) Patent Application Publication
(10) Pub. No.: US 2016/0015803 A1
(43) Pub. Date: Jan. 21, 2016

(54) IMMUNOSTIMULATORY COMBINATIONS AND USE THEREOF

(71) Applicant: Ross Kedl, Centennial, CO (US)

(72) Inventor: Ross Kedl, Centennial, CO (US)

(21) Appl. No.: 14/802,532

(22) Filed: Jul. 17, 2015

Related U.S. Application Data
(60) Provisional application No. 62/026,353, filed on Jul. 18, 2014.

(51) Int. Cl. A61K 39/21; A61K 39/395; C12N 7/00; C07K 16/28

(52) U.S. Cl. CPC A61K 39/21; C07K 16/2878; A61K 39/3955; C12N 7/00; A61K 2039/545

(57) ABSTRACT

Immunostimulatory combination are provided comprising: (i) a TLR agonist, (ii) a CD40 agonist, and (iii) an antigen, wherein said antigen consists of one or more antigenic peptides which range in size from 6-14 amino acids in length, wherein these moieties are each present in an amount that, in combination with the other, is effective to increase a subject's immune response to an antigen. The use of such short peptides unexpectedly enhances CD8⁺ T cell immunity.

### RELATED APPLICATIONS

This application claims priority to U.S. provisional Ser. No. 62/026,353, filed on Jul. 18, 2014, the contents of which are incorporated by reference in their entirety herein.

### BACKGROUND OF THE INVENTION

There has been a major effort in recent years, with significant success, to discover new molecules and drug compounds that act by stimulating certain key aspects of the immune system, as well as by suppressing certain other aspects. These compounds, referred to herein as immune response modifiers (IRMs), act through basic immune system mechanisms known as Toll-like receptors (TLRs) to induce selected cytokine biosynthesis. For example, certain IRMs may be useful for treating viral diseases (e.g., human papilloma virus, hepatitis, herpes), neoplasias (e.g., basal cell carcinoma, squamous cell carcinoma, actinic keratosis, melanoma), and TH2-mediated diseases (e.g., asthma, allergic rhinitis, atopic dermatitis, multiple sclerosis), and are also useful as vaccine adjuvants.

Many of the IRM compounds are small organic molecule imidazoquinoline amine derivatives (see, e.g., U.S. Pat. No. 4,689,338), but a number of other compound classes are known as well (see, e.g., U.S. Pat. Nos. 5,446,153; 6,194,425; and 6,110,929) and more are still being discovered. Other IRMs have higher molecular weights, such as oligonucleotides, including CpGs (see, e.g., U.S. Pat. No. 6,194,388).

In view of the great therapeutic potential for IRMs, and despite the important work that has already been done, there is a substantial ongoing need to expand their uses and therapeutic benefits. Is further known that certain combinations of IRMs may elicit a synergistic effect on immunity, especially Th1 immunity and CD4⁺ or CD8⁺ T cell immunity. In particular, the present inventor has previously disclosed that the combination of a TLR agonist and a CD40 agonist elicits a synergistic effect on TH1 and CD4⁺ or CD8⁺ T cell immunity.

### SUMMARY OF THE INVENTION

In one aspect, the invention provides immunostimulatory combinations that include (i) TLR agonist, (ii) a CD40 agonist, and (iii) one or more peptide antigens ranging in size from 6-14 amino acids, more preferably 7-13, 7-12, 7-11 amino acids, and most preferably 8-10 or 11-14 amino acids in length, each in an amount that, in combination with the other, which are effective for increasing the immune response by a subject against said at least one peptide antigen, especially a CD8⁺ or CD4⁺ T cell immune response. In some embodiments, the moieties in such immunostimulatory combination may be in the same composition. Alternatively, these moieties may be in different compositions for use in therapeutic regimens wherein promoting CD8⁺ or CD4⁺ immunity is therapeutically desired.

In another aspect, the invention provides methods of using immunostimulatory combinations that include (i) TLR agonist, (ii) a CD40 agonist, and (iii) one or more peptide antigens ranging in size from 6-14 amino acids, more preferably 7-13, 7-12, 7-11 amino acids, and most preferably 8-10 or 11-14 amino acids in length, each in an amount that, in combination with the other, which are effective for increasing the immune response by a subject against said at least one peptide antigen, especially a CD8⁺ or CD4⁺ T cell immune response in order to promote CD8⁺ or CD4⁺ T cell immunity in a subject in need thereof. In these methods the moieties in such immunostimulatory combination which are administered to a subject in need thereof may be in the same composition, or may be in different compositions, which are administered concurrently or these compositions may be administered at different times sufficiently proximate for synergy to be obtained, i.e., synergistic enhancement in CD8⁺ or CD4⁺ T cell immunity.

In a preferred aspect, the invention provides immunostimulatory combinations that include (i) a TLR agonist, (ii) a CD40 agonist, and (iii) one or more peptide antigens that are specific to a tumor or infectious agent antigen, e.g., a viral, bacterial, parasite, fungal or human tumor antigen ranging in size from 6-14 amino acids, more preferably 7-13, 7-12, 7-11 amino acids, and most preferably 8-10 or 11-14 amino acids in length, each in an amount that, in combination with the other, which are effective for increasing the immune response by a subject against said at least one peptide antigen, especially a CD8⁺ or CD4⁺ T cell immune response. Again, the moieties in such immunostimulatory combination may be in the same composition or in different compositions for use in therapeutic regimens wherein promoting CD8⁺ or CD4⁺ immunity is therapeutically desired.

In an especially preferred aspect the TLR agonist is poly-IC and the CD40 agonist is an agonistic anti-CD40 antibody or antibody fragment and the peptide antigens are specific to an antigen expressed by a virus or virally infected cells or a human tumor antigen, i.e., are short fragments thereof (at most 14 amino acids long).

However, before describing the invention in more detail the following definitions are provided. Otherwise all words and phrases herein are to be accorded their usual definition, as construed by a skilled artisan.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims. As used herein the singular forms “a”, “and”, and the include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

“Agonist” refers to a compound that, in combination with a receptor, can produce a cellular response. An agonist may be a ligand that directly binds to the receptor. Alternatively, an agonist may combine with a receptor indirectly by, for example, (a) forming a complex with another molecule that directly binds to the receptor, or (b) otherwise resulting in the modification of another compound so that the other compound directly binds to the receptor. An agonist may be referred to as an agonist of a particular receptor or family of receptors (e.g., a CD40 agonist or a Toll-like Receptor (TLR) member agonist).

“Antagonist” refers to a compound that when contacted with a molecule of interest, e.g. a TNF or TNFR family superfamily member or other ligand or receptor causes a decrease in the magnitude of a certain activity or function of the molecule compared to the magnitude of the activity or function observed in the absence of the antagonist.

“Antigen” refers to any substance that is capable of being the target of an immune response. An antigen may be the target of, for example, a cell-mediated and/or humoral immune response raised by a subject organism. Alternatively, an antigen may be the target of a cellular immune response (e.g., immune cell maturation, production of cytokines, production of antibodies, etc.) when contacted with immune cells. Herein, the preferred antigens are small peptides, i.e., a size of at most 6-14 amino acids, more preferably 7-13, 7-12, 7-11 and most preferably 11-14 or 8-10 amino acids long. These peptides will preferably comprise fragments of antigens expressed by of antigen to which a CD4⁺ or CD8⁺ immune response is desirably elicited against, e.g., tumor antigens or infectious agents such as viruses, bacteria, yeast and fungi, mycoplasma, and parasites. In general the antigens will be expressed on the surface of tumor cells or infectious agents or will be expressed on the surface of cells infected by an infectious agent. However, in some embodiments the antigens may be intracellular.

Co-administered” refers to two or more components of a combination administered so that the therapeutic or prophylactic effects of the combination can be greater than the therapeutic or prophylactic effects of either component administered alone. Two components may be co-administered simultaneously or sequentially. Simultaneously co-administered components may be provided in one or more pharmaceutical compositions. Sequential co-administration of two or more components includes cases in which the components are administered so that each component can be present at the treatment site at the same time. Alternatively, sequential co-administration of two components can include cases in which at least one component has been cleared from a treatment site, but at least one cellular effect of administering the component (e.g., cytokine production, activation of a certain cell population, etc.) persists at the treatment site until one or more additional components are administered to the treatment site. Thus, a co-administered combination can, in certain circumstances, include components that never exist in a chemical mixture with one another.

“Mixture” refers to any mixture, aqueous or non-aqueous solution, suspension, emulsion, gel, cream, or the like, that contains two or more components. The components may be, for example, two immunostimulatory components that, together, provide an immunostimulatory combination. The immunostimulatory components may be any combination of one or more antigens, one or more adjuvants, or both. For example, a mixture may include two adjuvants so that the mixture forms an adjuvant combination. Alternatively, a mixture may include an adjuvant combination and an antigen so that the mixture forms a vaccine.

“Synergy” and variations thereof refer to activity (e.g., immunostimulatory activity) of administering a combination of compounds that is greater than the additive activity of the compounds if administered individually.

“TLR” generally refers to any Toll-like receptor of any species of organism. A specific TLR may be identified with additional reference to species of origin (e.g., human, murine, etc.), a particular receptor (e.g., TLR6, TLR7, TLR8, etc.), or both.

“TLR agonist” refers to a compound that acts as an agonist of a TLR. Unless otherwise indicated, reference to a TLR agonist compound can include the compound in any pharmaceutically acceptable form, including any isomer (e.g., diastereomer or enantiomer), salt, solvate, polymorph, and the like. In particular, if a compound is optically active, reference to the compound can include each of the compound's enantiomers as well as racemic mixtures of the enantiomers. Also, a compound may be identified as an agonist of one or more particular TLRs (e.g., a TLR7 agonist, a TLR8 agonist, or a TLR7/8 agonist).

“CD40 agonist” refers to a compound that acts as an agonist of CD40, preferably human CD40. Examples thereof include agonistic anti-CD40 antibodies and antibody fragments and CD40L polypeptides and fragments and conjugates thereof, especially multimeric forms. Preferred examples thereof include agonistic anti-human CD40 antibodies and antibody fragments and human CD40L polypeptides and fragments and conjugates thereof, especially multimeric forms. These antibodies may comprise different isotypes, e.g., human IgG1, IgG2, IgG3 and IgG4's. Also, the antibody may comprise a constant region that is modified to increase or decrease an effector function such as FcR binding, FcRn binding, complement function, glycosylation, C1q binding; complement dependent cytotoxicity (CDC); Fc receptor binding; antibody-dependent cell-mediated cytotoxicity (ADCC); phagocytosis; down-regulation of cell surface receptors (e.g. B cell receptor; BCR), etc.

Examples of cancers treatable by the present invention include carcinoma, lymphoma, blastoma, sarcoma, and leukemia. More particular examples of such cancers include Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Müllerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sézary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenström's macroglobulinemia, Warthin's tumor, Wilms' tumor, or any combination thereof.

The present invention in particular may be used to treats B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenström's Macroglobulinemia); chronic lymphocytic leukemia (CLL); acute lymphoblastic leukemia (ALL); Hairy cell leukemia; chronic myeloblastic leukemia; multiple myeloma and post-transplant lymphoproliferative disorder (PTLD), melanoma, ovarian cancer, brain cancer, solid tumors, stomach cancer, oral cancers, testicular cancer, uterine cancer, scleroderma, bladder cancer, esophageal cancer, et al.

Other preferred cancers especially amenable for treatment according to the present invention include, but are not limited to, carcinoma, blastoma, sarcoma, and leukemia or lymphoid tumors and myeloma, melanoma, lymphomas, leukemias, ovarian cancer, breast cancer, lung cancer such as non-small lung cancer (NSLC), small cell lung cancer, mesothelioma, pancreatic cancer, head and neck cancer, brain cancer, solid tumors, colorectal cancer, stomach cancer, oral cancers, testicular cancer, uterine cancer, scleroderma, bladder cancer, esophageal cancer, colorectal cancer, rectal cancer, non-Hodgkin's lymphoma (NHL), renal cell cancer, prostate cancer, liver cancer, pancreatic cancer, soft-tissue sarcoma, Kaposi's sarcoma, carcinoid carcinoma, head and neck cancer, melanoma, ovarian cancer, mesothelioma, and multiple myeloma.

The cancer or tumor antigens (or fragments thereof ranging in size from 5-14 amino acids, 6-13, 7-12, 7-11 and most preferably 8-10 or 11-14 amino acids long) used in the present invention in particular may be derived from tumor antigens such as MAGE, MART-1/Melan-A, gp100, Dipeptidyl peptidase IV (DPPIV), adenosine deaminase-binding protein (ADAbp), cyclophilin b, Colorectal associated antigen (CRC)—C017-1A/GA733, Carcinoembryonic Antigen (CEA) and its antigenic epitopes CAP-1 and CAP-2, etv6, am11, Prostate Specific Antigen (PSA) and its antigenic epitopes PSA-1, PSA-2, and PSA-3, prostate-specific membrane antigen (PSMA), T-cell receptor/CD3-.xi. chain, MAGE-family of tumor antigens (e.g., MAGE-A 1, MAGE-A2, MAGE-A3, MAGE-A4, MAGE-A5, MAGE-A6, MAGE-A7, MAGE-A8, MAGE-A9, MAGE-A10, MAGE-A11, MAGE-A 12, MAGE-Xp2 (MAGE-B2), MAGE-Xp3 (MAGE-B3), MAGE-Xp4 (MAGE-B4), MAGE-C1, MAGE-C2, MAGE-C3, MAGE-C4, MAGE-05), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21 ras, RCAS1, .alpha.-fetoprotein, E-cadherin, .alpha.-catenin, .beta.-catenin, .gamma.-catenin, p120 ctn, gp100.sup.Pmel117, PRAME, NY-ESO-1, cdc27, adenomatous polyposis coli protein (APC), fodrin, Connexin 37, Ig-idiotype, p15, gp75, GM2 and GD2 gangliosides, viral products such as human papilloma virus proteins, Smad family of tumor antigens, Imp-1, PIA, EBV-encoded nuclear antigen (EBNA)-1, brain glycogen phosphorylase, SSX-1, SSX-2 (HOM-MEL-40), SSX-3, SSX4, SSX-5, SCP-1 and CT-7, and c-erbB-2.

Examples of viral infections treatable by the present invention include those caused by single or double stranded RNA and DNA viruses, which infect animals, humans and plants, such as retroviruses, poxviruses, immunodeficiency virus (HIV) infection, echovirus infection, parvovirus infection, rubella virus infection, papillomaviruses, congenital rubella infection, Epstein-Barr virus infection, mumps, adenovirus, AIDS, chicken pox, cytomegalovirus, dengue, feline leukemia, fowl plague, hepatitis A, hepatitis B, HSV-1, HSV-2, hog cholera, influenza A, influenza B, Japanese encephalitis, measles, parainfluenza, rabies, respiratory syncytial virus, rotavirus, wart, and yellow fever, adenovirus, a herpesvirus (e.g., HSV-I, HSV-II, CMV, or VZV), a poxvirus (e.g., an orthopoxvirus such as variola or vaccinia, or molluscum contagiosum), a picornavirus (e.g., rhinovirus or enterovirus), an orthomyxovirus (e.g., influenzavirus), a paramyxovirus (e.g., parainfluenzavirus, mumps virus, measles virus, and respiratory syncytial virus (RSV)), a coronavirus (e.g., SARS), a papovavirus (e.g., papillomaviruses, such as those that cause genital warts, common warts, or plantar warts), a hepadnavirus (e.g., hepatitis B virus), a flavivirus (e.g., hepatitis C virus or Dengue virus), or a retrovirus (e.g., a lentivirus such as HIV).

The viral antigens (or fragments thereof ranging in size from 5-14 amino acids, more preferably 6-13, 7-12, or 7-11 and most preferably 8-10 amino acids long) used in the present invention in particular may be derived from viral envelope, polymerase, gag, tat, transcriptase, and capsid proteins.

Other specific examples of viral infections treatable by the use of the subject immunostimulatory combination include Adeno associated virus group, Adenoviridae, Adenovirus, AIDS virus, Alpharetrovirus, Alphavirus, ALV related virus, Amapari virus, arbovirus, arbovirus C, arbovirus group A, arbovirus group B, Arenavirus group, Arterivirus, Astrovirus, baculovirus, bluetongue virus, Bolivian hemorrhagic fever virus, Boma disease virus, Borgore Virus, borna virus, bracovirus, Bromovirus, Burkitt's lymphoma virus, California encephalitis virus, common cold virus, congenital cytomegalovirus, contagious, ecthyma virus, contagious pustular dermatitis virus, Coronavirus, croup associated virus, Crypotovirus, cytomegalovirus, cytomegalovirus group, cytoplasmic polyhedrosis virus, Dengue, EB virus, Ebola virus, Ebola-like virus, echo virus, encephalitis virus, encephalomyocarditis group virus, encephalomyocarditis virus, Enterovirus, Entomopoxvirus, enzyme elevating virus, enzyme elevating virus (LDH), epidemic hemorrhagic fever virus, epizootic hemorrhagic disease virus, Epstein-Barr virus, foot and mouth disease virus, HCMV (human cytomegalovirus), helper virus, Hepadnavirus, hepatitis A virus, hepatitis B virus, hepatitis C virus, hepatitis D (delta) virus, hepatitis E virus, hepatitis F virus, hepatitis G virus, hepatitis nonA, nonB virus, hepatoencephalomyelitis reovirus Hepatovirus, herpes B Virus, herpes simplex virus, herpes simplex virus, 1 herpes simplex virus, herpesvirus, herpes zoster herpesvirus 7, adenovirus 2, human alpha herpesvirus 1, human alphaherpesvirus 2, human alphaherpesvirus 3, human B lymphotropic virus, human beta herpesvirus 5, human coronavirus, human foamy virus, human gamma herpesvirus 4, human gamma herpesvirus 6, human hepatitis A virus, human herpesvirus 1 group, human herpesvirus 2 group, human herpesvirus 3 group, human herpesvirus 4 group, human herpesvirus 6, human herpesvirus 8, human immunodeficiency virus, human immunodeficiency virus 1, human immunodeficiency virus 2, Human metapneumovirus hMPV, Human parainfluenza viruses, human papillomavirus, human T cell leukemia virus, human T cell leukemia virus I, human T cell leukemia virus II, human T cell leukemia virus III, human T cell lymphoma virus I, human T cell lymphoma virus II, human T cell lymphotropic virus type 1, human T cell lymphotropic virus type 2, human T lymphotropic virus I. human T lymphotropic virus II, human T lymphotropic virus III, ichnovirus, Ilarvirus, infantile gastroenteritis virus, infectious bovine rhinotracheitis virus, infectious haematopoietic necrosis virus, infectious pancreatic necrosis virus, influenza A virus, influenza B virus, influenza virus (unspecified), influenzavirus, (unspecified), influenzavirus A, influenzavirus B, influenzavirus C, influenzavirus D, influenzavirus pr8, iridovirus, Japanese B virus, Japanese encephalitis virus, Kaposi's sarcoma-associated herpesvirus, Korean hemorrhagic fever virus, Lentivirus, Leporipoxvirus, leukemia virus, leukovirus, lumpy skin disease virus, Luteovirus, Lymphadenopathy Associated Virus, Lymphocryptovirus, lymphocytic choriomeningitis virus, lymphoproliferative virus group, mammalian type B oncovirus group, mammalian type B retroviruses, mammalian type C retrovirus group, mammalian type D retroviruses, mammary tumor virus, measles virus, Merkel cell polyomavirus, myxoma virus, Myxovirus, disease virus, neurotropic virus, New World Arenavirus, newborn pneumonitis virus, Newcastle disease virus, noncytopathogenic virus, Norovirus, Norwalk virus, nuclear polyhedrosis virus (NPV), oncogenic virus, oncogenic virus like particle, oncornavirus, Orbivirus, Orf virus, orthomyxovirus, Orthopoxvirus, Orthoreovirus, Papillomavirus, Papillomavirus sylvilagi, Papovavirus, parainfluenza virus, Paramyxovirus, Parapoxvirus, paravaccinia virus, Parvovirus, Parvovirus, Picornavirus, Pneumovirus, poliomyelitis virus, poliovirus, polyoma virus, Polyomavirus, pox virus, provirus, pseudorabies virus, rabies virus, recombinant vaccinia virus, reovirus, respiratory infection virus, respiratory syncytial virus, respiratory virus, reticuloendotheliosis virus, Retrovirus, Rhabdovirus, Rhadinovirus, rhinovirus, Rhizidiovirus, rinderpest virus, RNA tumor virus, RNA virus, Rotavirus, Rous, sarcoma virus, rubella virus, rubeola virus, simian virus, Sabia virus, Sabio virus, Sabo virus, salivary gland virus, SARS virus, satellite virus, Sendai virus, Seoul virus, Simplexvirus, Sindbis virus, smallpox virus, submaxillary virus, Swine cytomegalovirus, Swine infertility and respiratory syndrome virus, swinepox virus, Triatoma virus, type C retroviruses, type D oncovirus, type D retrovirus group, Uasin Gishu disease virus, Uganda S virus, Ugymyia sericariae NPV, ulcerative disease rhabdovirus, Vaccinia virus, Varicella zoster virus, Varicellovirus, Varicola virus, Variola major virus, Variola virus, Vibrio phage, viral haemorrhagic septicemia virus, virus-like particle, Visna Maedi virus, Visna virus, West Nile virus, Western equine encephalitis virus, Western equine encephalomyelitis virus, and Yellow fever virus.

It is especially contemplated to treat “Human Immunodeficiency Virus” or “HIV” infection which refer to the disease caused by the HIV virus which results in the failure of the host immune system and development of Acquired Immunodeficiency Syndrome (AIDS).

Bacterial diseases treatable by the invention include by way of example, diseases resulting from infection by bacteria of, for example, the genus Escherichia, Enterobacter, Salmonella, Staphylococcus, Shigella, Listeria, Aerobacter, Helicobacter, Klebsiella, Proteus, Pseudomonas, Streptococcus, Chlamydia, Mycoplasma, Pneumococcus, Neisseria, Clostridium, Bacillus, Corynebacterium, Mycobacterium, Campylobacter, Vibrio, Serratia, Providencia, Chromobacterium, Brucella, Yersinia, Haemophilus, or Bordetella.

Other specific examples of bacterial infections treatable according to the invention include, but are not limited to, Bordetella pertussis (which may cause Pertussis), Borrelia burgdorferi, Brucella abortus, Brucella canis, Brucella melitensis, Brucella suis, Campylobacter jejuni, Chlamydia pneumonia, Chlamydia trachomatis, Chlamydophila psittaci, Clostridium botulinum, Clostridium difficile, Clostridium perfringens, Clostridium tetani (which may cause Tetanus), Corynebacterium diphtheriae (which may cause Diphtheria), Echinococcus (which may cause Echinococcal disease), Enterococcus faecalis, Enterococcus faecium, Escherichia coli (which may cause diarrhea, hemolytic uremic syndrome or urinary tract infection) such as Enterotoxigenic E. coli, Enteropathogenic E. coli, Enterohemorrhagic E. coli or Enteroaggregative E. coli, Francisella tularensis, Haemophilus influenzae (which may cause respiratory infections or meningitis), Helicobacter pylori (which may cause gastritis, peptic ulcer disease or gastric neoplasms), Legionella pneumophila, Leptospira interrogans, Listeria monocytogenes, Mycobacterium leprae, Mycobacterium tuberculosis (which may cause tuberculosis), Mycobacterium ulcerans, Mycoplasma pneumonia, Neisseria gonorrhoeae, Neisseria meningitides, Pneumococcus (which may cause meningitis, pneumonia, bacteremia or otitis media), Pseudomonas aeruginosa, Rickettsia rickettsia, Salmonella (which may cause food poisoning) such as, Salmonella bongo, Salmonella enterica, Salmonella subterranean, Salmonella typhi or Salmonella typhimurium, Shigella (which may cause shigellosis or gastroenteritis) such as Shigella sonnei, Staphylococcus aureus, Staphylococcus epidermidis, Staphylococcus saprophyticus, Streptococcus agalactiae, Streptococcus pneumonia, Streptococcus pyogenes, Treponema pallidum, Vibrio cholerae (which may cause cholera) or Yersinia pestis.

Examples of parasitic diseases treatable using the immunostimulatory combinations of the invention include but not limited to those caused by plasmodium (malaria), Amoebiasis, Enterobiasis, Babesiosis, Balantidiasis, Blastocystosis, Coccidia, Dientamoebiasis, Entamoeba, Giardiasis, Hookworm, Isosporiasis, Leishmaniasis, tapeworm, pneumocystis carnii pneumonia, leishmaniasis, Primary amoebic meningoencephalitis, Rhinosporidiosis, Sarcocystis, Toxoplasmosis, cryptosporidiosis, schistosomiasis, trypanosome or African trypanosomiasis or sleeping sickness infection, Chagas disease, Cestoda or tapeworm infection, Diphyllobothriasis, Echinococcosis, Hymenolepiasis, Taenia saginata, Taenia solium, Bertielliasis, Sparganosis, Clonorchiasis, liver fluke infection (such as lonorchis sinensis, Dicrocoelium dendriticum (lancet liver fluke), Microcoelium hospes, Fasciola hepatica (the “sheep liver fluke”), Fascioloides magna (the “giant liver fluke”), Fasciola gigantica, Fasciola jacksoni, Metorchis conjunctus, Metorchis albidus, Protofasciola robusta, Parafasciolopsis fasciomorphae, Opisthorchis viverrini (Southeast Asian liver fluke), Opisthorchis felineus (cat liver fluke) and Opisthorchis guayaquilensis), Paragonimiasis, Schistosomiasis, Schistosoma mansoni, Urinary schistosomiasis, Asian intestinal schistosomiasis, Swimmer's itch, Ancylostomiasis, Angiostrongyliasis, Anisakis, Ascaris lumbricoides, Baylisascaris procyonis, lymphatic filariasis, Guinea worm or Dracunculiasis, Dracunculus medinensis, Pinworm or Enterobiasis, Enterobius vermicularis, Enterobius gregorii, Halicephalobiasis, Halicephalobus gingivalis, Loa loa filariasis, Mansonelliasis, Filariasis, Mansonella streptocerca, River blindness or Onchocerciasis, Onchocerca volvulus, Strongyloidiasis or Parasitic pneumonia, Strongyloides stercoralis, Thelaziasis, Thelazia californiensis, Thelazia callipaeda, Amiota (Phortica) variegata, Phortica okadai, Toxocariasis, Toxocara canis, Toxocara cati, Trichinosis, Trichinella spiralis, Trichinella britovi, Trichinella nelsoni, Trichinella nativa, Whipworm, Trichuris trichiura, Trichuris vulpis, lephantiasis Lymphatic filariasis, Wuchereria bancrofti, Acanthocephaliasis, Archiacanthocephala, Moniliformis moniliformis, Halzoun Syndrome, Linguatula serrata, Myiasis, Oestroidea, Calliphoridae, Sarcophagidae, and Tunga penetrans.

Examples of fungal infections which may be treated according to the invention include Aspergillus spp., Coccidioides immitis, Cryptococcus neoformans, Candida albicans and other Candida spp., Blastomyces dermatidis, Histoplasma capsulatum, Chlamydia trachomatis, Nocardia spp., and Pneumocytis carinii.

Various other features and advantages of the present invention should become readily apparent with reference to the following detailed description, examples, claims and appended drawings. In several places throughout the specification, guidance is provided through lists of examples. In each instance, the recited list serves only as a representative group and should not be interpreted as an exclusive list.

### BRIEF DESCRIPTION OF THE DRAWINGS

FIG. 1 contains experimental data evidencing that immunization with an antigen in combination with a TLR agonist and anti-CD40 treatment (combined TLR/CD40-agonist immunization) induces potent CD8+ T cell expansion, eliciting a response 10-20 fold higher than immunization with either agonist alone (FIG. 1A).

FIG. 2 contains experimental data evidencing that combined TLR/CD40 vaccination elicits both CD4 and CD8+ T cell responses at a magnitude normally only seen against infectious agents.

FIG. 3 contains experimental data evidencing that combined PolyIC-LC/CD40 vaccination is superior to use of antiCD40 or polyIC as a vaccine adjuvant alone.

FIG. 4 contains experimental data evidencing that combined PolyIC-LC/αCD40 vaccination produces an unparalleled magnitude of cellular immunity after a single immunization.

FIG. 5 contains experimental data evidencing that contrary to expectation and published data, that shorter peptides generated robust CD8⁺ T cell responses, while longer peptide sequences did not.

### DETAILED DESCRIPTION OF THE INVENTION

The generation of successful long-term immunity, both prophylactic and therapeutic, is the object of modern vaccine development. Much effort has gone into discovering the “adjuvants” present within infectious agents that stimulate the molecular pathways involved in creating the appropriate immunogenic context of antigen presentation. Numerous innate receptor pathways have been identified and have been targeted for use in modern vaccine adjuvant formulations. The central role played by TLRs in triggering innate immunity is mirrored by CD40 in controlling acquired immune responses. However, the use of adjuvants that target either these innate receptors or CD40 alone results in minimal expansion of antigen specific T cells (FIG. 1). A broad spectrum of clinical data also support the conclusion that TLR or CD40 stimulation alone (FIG. 1) fails to reconstitute all necessary signals for long term T cell immunity.

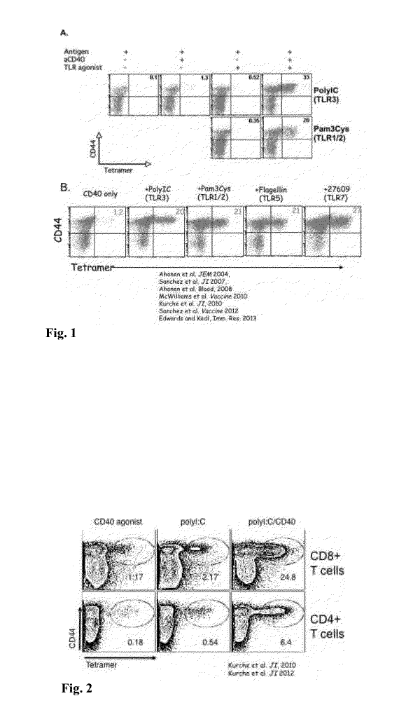

We have previously shown that immunization with antigen in combination with a TLR agonist and anti-CD40 treatment (combined TLR/CD40-agonist immunization) induces potent CD8+ T cell expansion, eliciting a response 10-20 fold higher than immunization with either agonist alone (FIG. 1A). Antigen-specific CD8+ T cells elicited by combined TLR/CD40-agonist immunization demonstrate lytic function, IFNγ production, and secondary response to antigenic challenge. Synergistic activity with anti-CD40 in the induction of CD8+ T cell expansion is a property of all TLR agonists examined which now includes agonists for TLRs 1/2, 2/6, 3, 4, 5, 7, and 9 (FIG. 1B). Further, combined TLR/CD40 vaccination elicits both CD4 and CD8+ T cell responses at a magnitude normally only seen against infectious agents (FIG. 2). Collectively, these data demonstrate that combined TLR/CD40-agonist immunization can reconstitute all of the signals required to elicit potent primary CD4+ and CD8+ T cell responses.

Based on these findings in mice we explored the potential for the use of polyIC-LC/αCD40 combination vaccination in non-human primates (NHP) in order to validate the clinical efficacy. Human anti-human CD40 (Kirin Pharmaceuticals) having the variable heavy and light sequences in the Sequence Listing were produced in cell culture. CD40 binding activity and agonistic activity was confirmed against both human and Rhesus Macaque PBMCs. In vivo activity in Rhesus was also confirmed by examining markers of innate activation 24-72 hours after IV antibody injection. PolyIC-LC was obtained from Oncovir Inc. and its activity was confirmed in vivo similar to the αCD40.

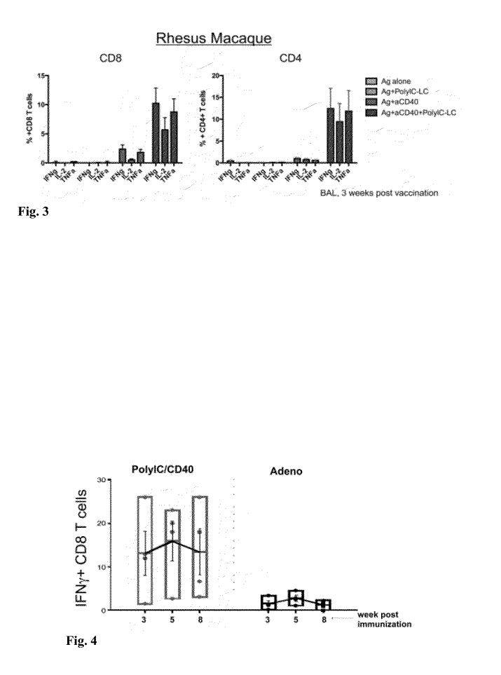

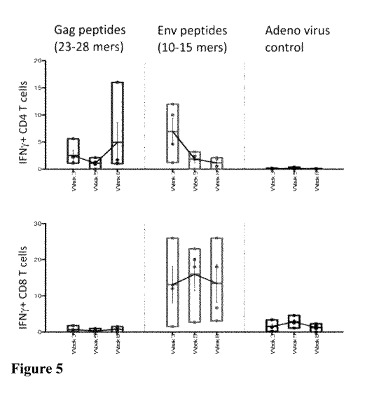

These experiments in the NHPs allowed us to form important conclusions relevant to the adjuvants of the present invention, i.e., that: 1) Combined PolyIC-LC/CD40 vaccination is superior to use of an agonistic anti-CD40 antibody or polyIC as a vaccine adjuvant alone (FIG. 3). Three weeks after vaccination, a bronchioalveolar lavage (BAL) sample (as a representative peripheral tissue) was obtained from the NHPs and analyzed for the presence of Env-specific CD4⁺ and CD8⁺ T cells by measuring intracellular IFNγ production. The data show that while detectable responses are observed in response to either adjuvant component alone, a substantial increase (˜8-10 fold) in both CD4⁺ and CD8⁺ T cell responses is seen in response to combined PolyIC-LC/αCD40 vaccination. These data corroborate the substantial amount of mouse data we have obtained over the years and confirm the synergistic impact of the combined vaccine adjuvant in higher primates. 2) Combined PolyIC-LC/αCD40 vaccination produces an unparalleled magnitude of cellular immunity after a single immunization (FIG. 4).

Typical vaccine schedules have the capacity to produce 1-5% antigen specific T cells within the BAL after 3-4 vaccinations. Comparing our vaccine platform to adenovirus challenge, typically used as the gold standard+ control for the generation of CD8⁺ T cell responses in vivo, again shows at least a 10 fold greater response in both CD4+ and CD8⁺ T cells in response to the vaccination. 3) The largest responses to vaccination are observed within the peripheral tissues (BAL) rather than in the peripheral blood (not shown). Since the intended target of the vaccination usually resides within peripheral/mucosal tissues, this represents a significant advantage of the present adjuvants.

In these experiments we assayed the effects of the form of the antigen and routes of administration. In the process of performing these experiments, we obtained a highly unanticipated result with regard to the form of antigen that is suitable for the generation of enhanced CD4⁺ and CD8⁺ responses. In particular, we surprisingly discovered that immunization with a TLR agonist, a CD40 agonist and short antigenic peptides, i.e. around 5-14 amino acids long, more preferably 6-13, or 7-12 or 7-11 amino acids long and most preferably 8-10 or 11-14 amino acids long, surprisingly greatly favors the generation of antigen-specific CD8+ or CD4+ immune responses, and most especially better CD8⁺ immune responses relative to when CD40 and TLR agonists were administered in association with antigens of larger size. This result was unanticipated as it was contrary to our own experience and our understanding of the scientific literature.

Previous publications have demonstrated that use of antigen in the form of long peptides (15-50mer) were better at generating both CD4⁺ and CD8⁺ T cell responses in vivo when compared to the use of shorter peptides comprised only of the epitope sequence. The rationale for these results was that immunization with an 8-10mer class I binding epitope would result in the loading of that peptide into the surface class I molecules of both professional APCs as well as non-APC parenchymal cells. The encounter of antigen specific T cells with their antigen on non-APCs was proposed to have a dampening, or tolerizing, effect on the T cell response to the antigen. It was rationalized that the inclusion of the CD8 epitope within a longer peptide would require antigen processing prior to presentation. As antigen processing is a function unique to professional APCs, it was believed that this would ensure that the class I binding epitope embedded within the larger sequence would only be presented on professional APCs.

With this being the state of the art, we fully anticipated that the use of longer peptide antigens would have favored CD8+ T cell responses. Indeed, based on the literature, we might have expected that the longer peptides would enhance CD8⁺ T cell responses as they would be even less likely to be aberrantly presented by non-APCs. However, as shown in the Example that follows the exact opposite was the case.

### Example 1

We performed an experiment using 2 different pools of peptides derived from HIV. One pool, derived from Gag sequences spanning a number of HIV clades (Table 1), was comprised of 7 peptides ranging in length from 23-28mers. The second pool, derived from HIV Env sequences (table 1), comprised of 7 peptides ranging in length from 9-14mers. NHPs were immunized with 3 or 8 mg/kg doses of all peptides within a specific pool in combination with 1 mg/kg polyIC:LC and 1 mg/kg anti-CD40 antibody. CD4+ and CD8+ T cell responses in the BAL were analyzed by ICCS and ELISPOT at 3, 5, and 8 weeks after immunization. Consistent with predictions, CD4+ T cell responses were generated well by both sets of peptides. Surprisingly however, and contrary to expectation and published data, we found that while the shorter peptides generated very robust CD8+ T cell responses, whereas the longer peptide sequences did not (FIG. 5).

We conclude from these data that CD8⁺ T cell responses in vivo in NHPs are unexpectedly biased toward the use of antigens with shorter sequences. The size of the longer peptides utilized (11-14mers) are predicted to not bind directly into class I MHC and thus must require some amount of processing in order to stimulate antigen specific CD8+ T cell responses. Exactly what form of processing this takes is unclear at the moment, However, the data suggest that this processing can modify only a limited number of residues and thus can only facilitate class I restricted responses to peptides of shorter length. This is again unexpected as there are no literature to rely upon that would have predicted this outcome.

### Adminstration

As noted the TLR agonist, CD40 agonist and short peptide antigens are provided (or administered, as appropriate to the form of the immunostimulatory combination) in an amount effective to increase the CD8+ T cell immune response to the short peptide antigens. For example, the TLR agonist can be administered in an amount from about 100 ng/kg to about 100 mg/kg. In some embodiments, the TLR agonist is administered in an amount from about 1 mg/kg to about 5 mg/kg. The particular amount of TLR agonist that constitutes an amount effective to increase the immune response to a particular antigen, however, depends to some extent upon certain factors including but not limited to the particular TLR agonist being administered; the particular antigen being administered and the amount thereof; the particular CD40 agonist being administered and the amount thereof; the state of the immune system (e.g., suppressed, compromised, stimulated); the method and order of administration of the TLR agonist, the CD40 agonist, and the antigen; the species to which the formulation is being administered; and the desired therapeutic result. Accordingly it is not practical to set forth generally the amount that constitutes an effective amount of the TLR agonist. Those of ordinary skill in the art, however, can readily determine the appropriate amount with due consideration of such factors.

Also, for example, the CD40 agonist may be administered in an amount from about 100 ng/kg to about 100 mg/kg. In certain embodiments, the CD40 agonist is administered in an amount from about 10 μg/kg to about 10 mg/kg. In some embodiments, the CD40 agonist is administered in an amount from about 1 mg/kg to about 5 mg/kg. The particular amount of CD40 agonist that constitutes an amount effective to increase the immune response to a particular antigen, however, depends to some extent upon certain factors including but not limited to the particular CD40 agonist being administered; the particular TLR agonist being administered and the amount thereof; the particular antigen being administered and the amount thereof; the state of the immune system; the method and order of administration of the TLR agonist, the CD40 agonist, and the antigen; the species to which the formulation is being administered; and the desired therapeutic result. Accordingly it is not practical to set forth generally the amount that constitutes an effective amount of the CD40 agonist. Those of ordinary skill in the art, however, can readily determine the appropriate amount with due consideration of such factors.

In some embodiments, the immunostimulatory combination may further include an antigen. When present in the immunostimulatory combination, the antigen may be administered in an amount that, in combination with the other components of the combination, is effective to generate an immune response against the antigen. For example, the antigen can be administered in an amount from about 100 ng/kg to about 100 mg/kg. In many embodiments, the antigen may be administered in an amount from about 10 μg/kg to about 10 mg/kg. In some embodiments, the antigen may be administered in an amount from about 1 mg/kg to about 5 mg/kg. The particular amount of antigen that constitutes an amount effective to generate an immune response, however, depends to some extent upon certain factors such as, for example, the particular antigen being administered; the particular TLR agonist being administered and the amount thereof; the particular CD40 agonist being administered and the amount thereof; the state of the immune system; the method and order of administration of the TLR agonist, the CD40 agonist, and the antigen; the species to which the formulation is being administered; and the desired therapeutic result. Accordingly, it is not practical to set forth generally the amount that constitutes an effective amount of the antigen. Those of ordinary skill in the art, however, can readily determine the appropriate amount with due consideration of such factors.

When present, the antigen may be administered simultaneously or sequentially with any component of the immunostimulatory combination. Thus, the antigen may be administered alone or in a mixture with one or more adjuvants (including, e.g., a TLR agonist, a CD40 agonist, or both). In some embodiments, an antigen may be administered simultaneously (e.g., in a mixture) with respect to one adjuvant, but sequentially with respect to one or more additional adjuvants.

Sequential co-administration of an antigen and other components of an immunostimulatory combination can include cases in which the antigen and at least one other component of the immunostimulatory combination are administered so that each is present at the treatment site at the same time, even though the antigen and the other component are not administered simultaneously. Sequential co-administration of the antigen and the other components of the immunostimulatory combination also can include cases in which the antigen or at least one of the other components of the immunostimulatory combination is cleared from a treatment site, but at least one cellular effect of the cleared antigen or other component (e.g., cytokine production, activation of a certain cell population, etc.) persists at the treatment site at least until one or more additional components of the combination are administered to the treatment site. Thus, it may be possible that an immunostimulatory combination of the invention can, in certain circumstances, include one or more components that never exist in a mixture with another component of the combination. These moieties may be administered by well-known means for administered immunological agents including e.g., orally, intravenously, subcutaneously, intraarterially, intramuscularly, intracardially, intraspinally, intratracheally, intrathoracically, intraperitoneally, intraventricularly, sublingually, transdermally, intramucosally, and/or via inhalation. Administration may be systemic, e.g. intravenously, or localized. Preferably the subject immunostimulatory combination is administered by intravenous, subcutaneous, intramuscular, intramucosal or other injection route.

SEQUENCE LISTING

Heavy Chain (SEQ ID NO: 1)
MSVSFLIFLPVLGLPWGVLSQVQLQQSGPGLVKPSQTLSLTCAISGD
SVSSNSATWNWIRQSPSRDLEWLGRTYYRSKWYRDYVGSVKSRIIIN
PDTSNNQFSLQLNSVTPEDTAIYYCTRAQWLGGDYPYYYSMDVWGQG
TTVTVSSASTKGPSVFPLAPCSRSTSESTAALGCLVKDYFPEPVTVS
WNSGALTSGVHTFPAVLQSSGLYSLSSVVTVPSSNFGTQTYTCNVDH
KPSNTKVDKTVERKCCVECPPCPAPPVAGPSVFLFPPKPKDTLMISR
TPEVTCVVVDVSHEDPEVQFNWYVDGVEVHNAKTKPREEQFNSTFRV
VSVLTVVHQDWLNGKEYKCKVSNKGLPAPIEKTISKTKGQPREPQVY
TLPPSREEMTKNQVSLTCLVKGFYPSDIAVEWESNGQPENNYKTTPP
MLDSDGSFFLYSKLTVDKSRWQQGNVFSCSVMHEALHNHYTQKSLSL
SPGK
Light Chain (SEQ ID NO: 2)
MEAPAQLLFLLLLWLPDTTGEIVLTQSPATLSSLPGERATLSCRASQ
SVSSYLAWYQQKPGQAPRLLIYDASNRATGIPARFSGSGSGTDFTLT
ISSLEPEDFAVYYCQQRSNTFGPGTKVDIKRTVAAPSVFIFPPSDEQ
LKSGTASVVCLLNNFYPREAKVQWKVDNALQSGNSQESVTEQDSKDS
TYSLSSTLTLSKADYEKHKVYACEVTHQGLSSPVTKSFNRGE C
red = signal sequence.
Blue = variable region.
Black = constant region.
Heavy chain is constant region of IgG2. Contains
proline-serine switch at residue 331 (purple,
underline, bold) to inhibit Fc binding and
consequent ADCC.

TABLE 1

| HIV Gag | HIV Env |
|---|---|
| 23-28mer peptides | 9-14mer peptides |
| MEEKAFSPEVIPMFTALSEGATPQDLN | YLRDQQLLGIWG |
| LSEGATPQDLNTMLNTVGGHQAAMQML | RQQQNNLLRAIEA |
| LNTVGGHQAAMQMLKDTINEEAAEWDR | VYYGVPVWKEA |
| IYKRWIILGLNKIVRMYSPVSILDIR | LWDQSLKPCVKLT |
| RMYSPVSILDIRQGPKEPFRDYVDRF | SVITQACSKVSFE |
| ARNCRAPRKKGCWKCGKEGHQMKDCT | GTGPCTNVSTVQC |
| KCGKEGHQMKDCTERQANFLGKIWPS | YKVVKIEPL |
| Le tourneau et al. 2007 | Nehete et al. 2008 |
| PLOS1.10:984 | Virology 370:130 |

1. An immunostimulatory combination comprising: (i) a TLR agonist, (ii) a CD40 agonist, and (iii) an antigen, wherein said antigen consists of one or more antigenic peptides which range in size from 6-14 amino acids in length, wherein these moieties are each present in an amount that, in combination with the other, is effective to increase a subject's immune response to an antigen.

2. The immunostimulatory combination of claim 1, wherein said antigenic peptides range in size from 6-14, 7-11, 11-14, or 8-10 amino acids in length.

3. The immunostimulatory combination of claim 1, wherein said antigenic peptides range in size from 11-14 or 8-10 amino acids in length.

4. The immunostimulatory combination of claim 1 wherein said antigenic peptides comprise viral, bacterial, fungal tumor, or parasite antigens.

5. The immunostimulatory combination of claim 1 wherein said antigenic peptides comprise fragments of one or more viral, bacterial, fungal tumor, or parasite antigens and/or are expressed on the surface of a virus, bacterium, fungus, parasite, or tumor cell or a cell infected by a virus, bacterium, fungus, parasite.

6-12. (canceled)

13. The immunostimulatory combination of claim 1 wherein the peptide antigen is contained within an antigen expressed by a human tumor.

14. (canceled)

15. The immunostimulatory combination of claim 1 wherein the TLR agonist is poly-IC or flagellin.

16. The immunostimulatory combination of claim 1 wherein the CD40 agonist is an agonistic anti-CD40 antibody or antibody fragment or comprises a CD40L polypeptide or conjugate containing a CD40L polypeptide.

17. The immunostimulatory combination of claim 1 wherein the CD40 agonist comprises an agonistic anti-CD40 antibody having the heavy and light chain sequences of SEQ ID NO:1 and 2 or an antibody comprising the same CDRs.

18. The immunostimulatory combination of claim 1 wherein the TLR agonist is an agonist of at least one of TLR1, TLR2, TLR3, TLR4, TLR5, TLR6, TLR7, TLR8, TLR9, TLR10, or any combination of any of the foregoing.

19. The immunostimulatory combination of claim 1, wherein the antigenic peptides consist of any combination of the 9-14mer peptides identified in Table 1.

20. A method of promoting CD8⁺ T cell immunity by administering an effective amount of an immunostimulatory combination according to claim 1, wherein the moieties may be in the same or different formulations and may be separately or jointly administered at the same or different dosing frequencies.

21. The method of claim 20, which is used to treat cancer or infection.

22. (canceled)

23. The method of claim 20, which is used to treat viral infection.

24. The method of claim 20, which is used to treat bacterial infection.

25. The method of claim 20, which is used to treat parasite infection.

26. The method of claim 20, which is used to treat fungal or yeast infection.

27. The method of claim 20, wherein the TLR agonist is polyIC, the CD40 agonist is an agonistic CD40 antibody, and the antigens comprise HIV antigenic peptides.

28. The method of claim 27, wherein the peptides are fragments of HIV ENV, POL, or TAT antigens.

29. The method of claim 27, wherein the HIV antigenic peptides consist of any combination of the 9-14mer peptides identified in Table 1.


SEQUENCE LISTING (2 sequences)

SEQ ID NO: 1 - protein, 474 aa, Artificial sequence
  Heavy Chain
MSVSFLIFLPVLGLPWGVLSQVQLQQSGPGLVKPSQTLSLTCAISGDSVSSNSATWNWIRQSPSRDLEWLGRTYYRSKWYRDYVGSVKSRIIINPDTSNNQFSLQLNSVTPEDTAIYYCTRAQWLGGDYPYYYSMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 2 - protein, 231 aa, Artificial Sequence
  Light Chain
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQRSNTFGPGTKVDIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC